(19) United States
(12) Patent Application Publication
(10) Pub. No.: US 2024/0349989 A1
(43) Pub. Date: Oct. 24, 2024

(54) FAIRY LIGHT ENDOSCOPE FOR USE IN GASTROINTESTINAL (GI) MEDICAL PRACTICE

(71) Applicants: JOEL RILEY, ST. LOUIS, MO (US); EVE RILEY, ST. LOUIS, MO (US)

(72) Inventors: JOEL RILEY, ST. LOUIS, MO (US); EVE RILEY, ST. LOUIS, MO (US)

(21) Appl. No.: 18/609,502

(22) Filed: Mar. 19, 2024

Related U.S. Application Data
(60) Provisional application No. 63/461,139, filed on Apr. 21, 2023.

(51) Int. Cl. A61B 1/00; A61B 1/005

(52) U.S. Cl. CPC A61B 1/00096; A61B 1/005

(57) ABSTRACT

An endoscope has a distal end surface with pairs of white light lenses and ultraviolet light lenses having convex surface configurations positioned on opposite sides of an objective lens. The pairs of white light lenses and ultraviolet light lenses provide a wide area of illumination adjacent to the distal end surface of the endoscope being observed by the objective lens. A yellow light lens having a convex surface configuration is also positioned on the distal end surface of the endoscope in line with the objective lens.

### CROSS-REFERENCE TO RELATED APPLICATIONS

This application claims the benefit of the filing date of U.S. Provisional Patent Application No. 63/461,139, which was filed on Apr. 21, 2023, and is incorporated herein by reference.

### BACKGROUND OF THE INVENTION

FIG. 1 is a representation of a conventional, prior art endoscopy system 2. The endoscopy system 2 has three primary parts, a manual control 4, an insertion tube 6 and a video system 8.

The manual control 4 is connected to the insertion tube 6. The insertion tube 6 is basically a flexible tube with a distal end tip. A series of wires extend from the distal end tip through the insertion tube 6 to the manual control 4. There are knobs and other control devices on the manual control 4 that can be manually operated to cause movements of the wires extending through the insertion tube 6 that in turn control movements of the distal end tip of the insertion tube.

FIG. 2 is a representation of the end surface of the distal end tip of a conventional, prior art endoscope such as that represented in FIG. 1. As represented in FIG. 2, there is an objective lens 10 positioned on the distal end surface 12 of the endoscope 14. A conventional image sensor is contained inside the distal end tip of the insertion tube 6. For example, a solid-state CCD image sensor is contained in the distal end tip behind the objective lens 10. A photosensitive surface of the image sensor is positioned just behind the objective lens 10. The objective lens 10 focuses images of the environment being viewed by the endoscope, and specifically by the objective lens, on the photosensitive surface of the image sensor. The image sensor communicates the sensed images through electrical wires extending through the insertion tube 6, through the manual control 4 and through a universal cord to a video processor of the video system 8. The video processor displays images on a display screen of the video system 8 for the medical professional using the endoscope. The images displayed on the video screen are those sensed by the objective lens 10.

There are also light emitting lenses 16, 18, for example white light lenses positioned on the distal end surface 12 of the endoscope 14. The white light lenses 16, 18 illuminate the environment being viewed by the objective lens 10 of the endoscope.

The conventional endoscope also typically has a water nozzle opening 22, a water jet opening 24, an air nozzle 26 and an opening of an instrument channel 28 emerging from the distal end surface 12 of the endoscope 14.

As represented in FIG. 2, the distal end surface 12 of the conventional endoscope 14 is generally flat. The light emitting surfaces of the white light lenses 16, 18 are also generally flat. Light emitted from the white light lenses 16, 18 is emitted directly in front of the distal end surface 12. The illumination of the environment being viewed by the white light lenses 16, 18 is limited to the environment directly in front of the white light lens 16, 18.

Furthermore, the illumination of the environment being viewed is provided by only white light. Certain features of the environment being viewed may not be illuminated by white light. For example, neoplasms are often not sufficiently illuminated and are not visible to the objective lens 10 of an endoscope under white light alone.

### SUMMARY OF THE INVENTION

This disclosure pertains to an endoscope having a flexible length and a distal end surface with a plurality of light emitting lenses relatively positioned on the distal end surface. More specifically, this disclosure pertains to an endoscope having a distal end surface with pairs of white light lenses and pairs of ultraviolet light lenses having convex surface configurations that are positioned on opposite sides of an objective lens, and a yellow light lens having a convex surface configuration that is positioned on the distal end surface of the endoscope in line with the objective lens.

The endoscope has an insertion tube with a flexible length. There is a distal end tip at one end of the length of the insertion tube. The distal end tip has a distal end surface. The distal end surface has a circular configuration with a center point of the circular configuration. A first center line extends across the distal end surface and intersects the center point. A second center line also extends across the distal end surface and intersects the center point. The first and second center lines are mutually perpendicular.

An objective lens is positioned on the distal end surface. The objective lens is positioned off center of the center point, to one side of the center point and to one side of the first center line. The objective lens has a circular configuration and a generally planar exterior surface. However, the objective lens could have a slightly convex or slightly domed exterior surface configuration. The objective lens is positioned on the distal end surface centered along the second center line of the distal end surface.

A pair of white light lenses are positioned on the distal end surface on opposite sides of the objective lens. The pair of white light lenses are positioned to one side of the first center line and on opposite sides and equally spaced from the second center line. Each of the white light lenses has an exterior surface with a convex or domed configuration, meaning that the exterior surfaces of the pair of white light lenses have a greater convexity or greater domed configuration compared to that of the objective lens.

A pair of ultraviolet light lenses are positioned on the distal end surface on opposite sides of the objective lens. The pair of ultraviolet light lenses are positioned to one side of the first center line and on opposite sides and equally spaced from the second center line. Each of the ultraviolet light lenses has an exterior surface with a convex or domed configuration, meaning that the exterior surfaces of the pair of ultraviolet light lenses have a greater convexity or greater domed configuration compared to that of the objective lens.

There is also a yellow light lens positioned on the distal end surface along the second center line. The yellow light lens is positioned on the opposite side of the first center line from the objective lens, from the pair of white light lenses and from the pair of ultraviolet light lenses. The yellow light lens has an exterior surface with a convex or domed configuration, meaning that the exterior surface of the yellow light lens has a greater convexity or greater domed configuration compared to that of the objective lens.

The features, functions and advantages that have been discussed can be achieved independently in various embodiments or may be combined in yet other embodiments, further details of which can be seen with reference to the following description and drawings.

### BRIEF DESCRIPTION OF DRAWINGS

Further objects and features of the improved endoscope for use in gastrointestinal medical practice of this disclosure are set forth in the following detailed description of the invention and in the drawing figures.

FIG. 1 is a representation of a conventional, prior art endoscopy system.

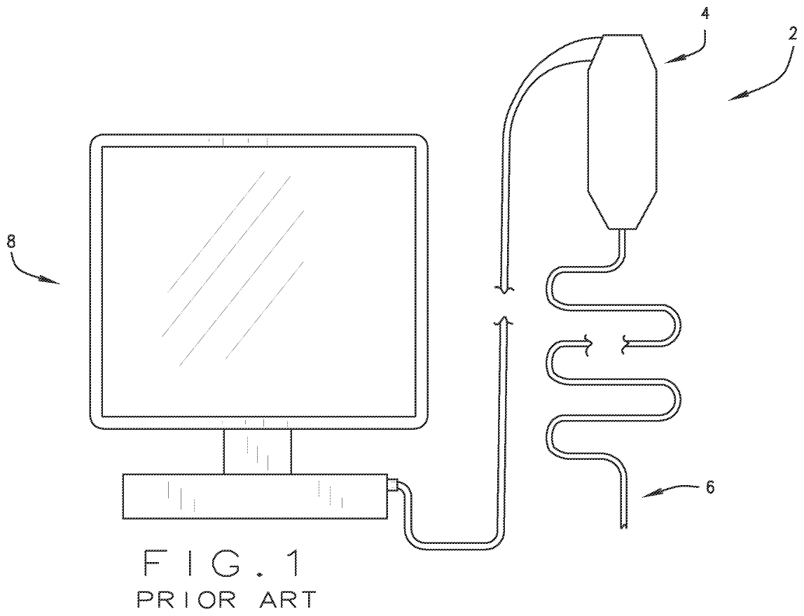

FIG. 2 is a representation of a distal end surface of a prior art endoscope and the relative positioning of lenses on the distal end surface.

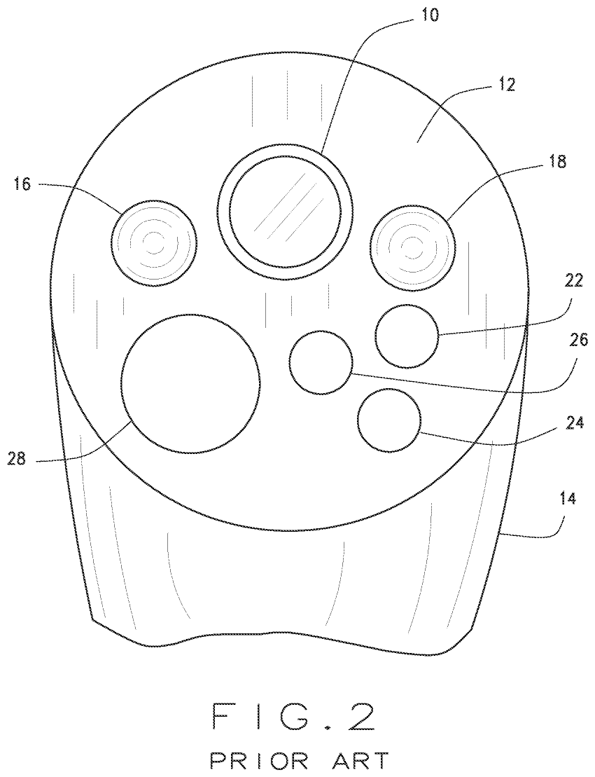

FIG. 3 is a representation of the distal end surface and relative positions of the objective lens and the light lenses of the endoscope of this disclosure.

FIG. 4 is a representation of a perspective view of the distal end surface of the endoscope of FIG. 3.

FIG. 5 is a representation of a side elevation view of the distal end of the endoscope of FIG. 3.

### DETAILED DESCRIPTION OF THE INVENTION

The above disadvantages of a conventional endoscope are overcome by the improved and novel construction of the endoscope of this disclosure.

The endoscope of this disclosure includes several known features of a prior art endoscopy system. The endoscope is comprised of an insertion tube having a flexible length with opposite proximal and distal ends. A manual control is connected with the proximal end of the insertion tube and a universal cord connects the manual control with a video system. The novel features of this disclosure are provided at the distal end of the insertion tube.

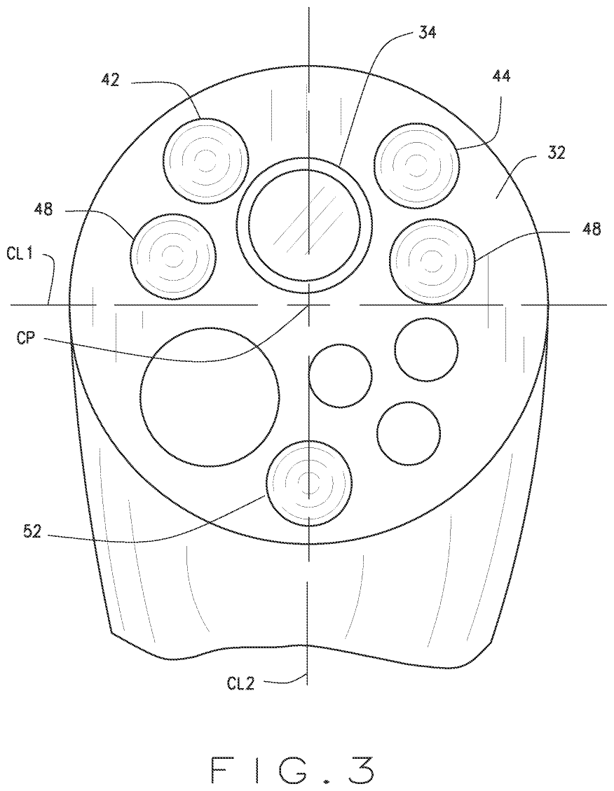

As represented in FIGS. 3-5, the distal end of the insertion tube has a distal end surface 32. As represented, the distal end surface 32 is a planar surface with a circular configuration. The circular configuration of the distal end surface 32 has a center point CP. A first center line CL1 extends across the distal end surface 32 and through the center point CP. A second center line CL2 extends across the distal end surface 32 and through the center point CP. The first center line CL1 and the second center line CL2 are mutually perpendicular.

An objective lens 34 is positioned on the distal end surface 32. As explained earlier, the objective lens is positioned in front of an image sensor contained inside the distal end of the insertion tube. The objective lens 34 has a circular configuration and a generally flat or planar exterior surface. However, the objective lens 34 could have a slightly convex surface configuration. The objective lens 34 is positioned to one side of the distal end surface 32 from the center point CP and to one side of or above the first center line CL1 as represented in FIG. 3. The objective lens 34 is positioned on the distal end surface 32 centered along the second center line CL2 of the distal end surface.

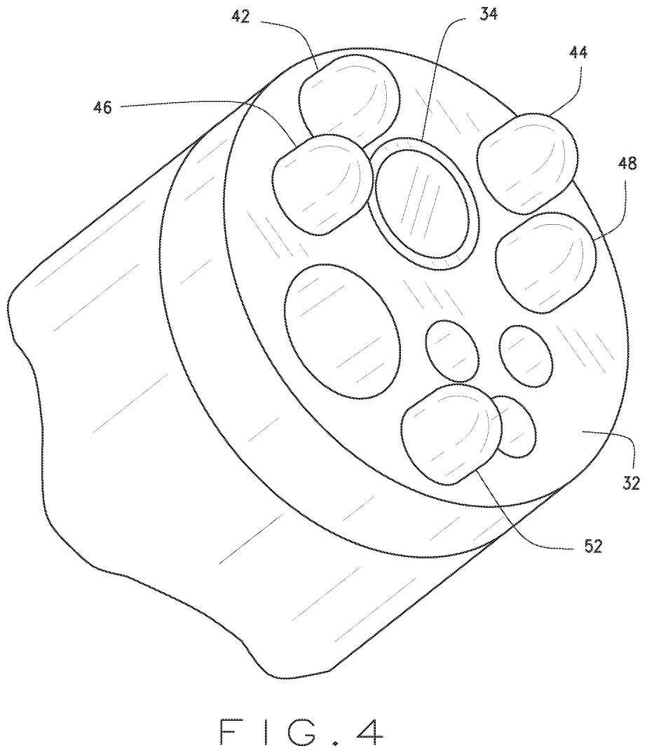

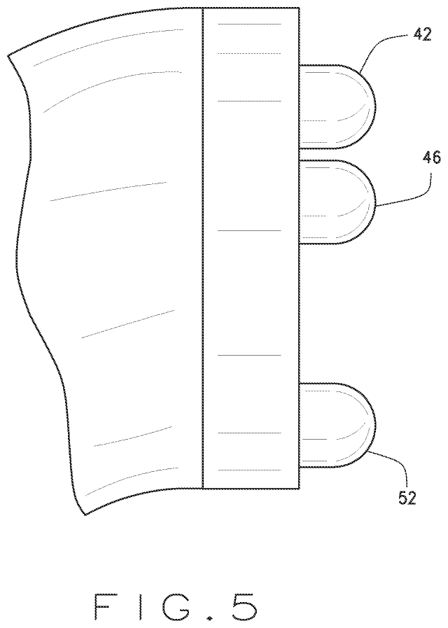

A pair of white light emitting lenses or white light lenses 42, 44 are positioned on the distal end surface 32. The pair of white light lenses 42, 44 are positioned on the distal end surface 32 on opposite sides of the objective lens 34, to one side of or above the first center line CL1 and on opposite sides and equally spaced from the second center line CL2. As represented in FIGS. 4 and 5, each of the white light lenses 42, 44 has an exterior surface with a convex or domed configuration. The convex or domed configurations of the white light lenses 42, 44 is comparatively greater than any convex configuration of the objective lens 34. This configuration of the white light lenses 42, 44 enhances their ability to illuminate a wide area of the environment adjacent to the distal end surface 32 that is observed by the objective lens 34.

A pair of ultraviolet light emitting lenses or ultraviolet light lenses 46, 48 are also provided on the distal end surface 32. The pair of ultraviolet light lenses 46, 48 are positioned on the distal end surface 32 on opposite sides of the objective lens 34 and are equally spaced on opposite sides of the second center line CL2. The pair of ultraviolet light lenses 46, 48 are also positioned on the distal end surface 32 to one side of or above the first center line CL1. As represented in FIGS. 4 and 5, each of the ultraviolet light lenses 46, 48 has an exterior surface with a convex or domed configuration. The convex or domed configurations of the ultraviolet light lenses 46, 48 is comparatively greater than any convex configuration of the objective lens 34. This configuration of the ultraviolet light lenses 46, 48 enhances their ability to illuminate a wide area of the environment adjacent to the distal end surface 32 that is being observed by the objective lens 34.

As represented in FIG. 3, positioning the objective lens 34, the pair of white light lenses 42, 44 and the pair of ultraviolet light lenses 46, 48 all above the center point CP and all above the first center line CL1 clusters the white light lenses 42, 44 and the ultraviolet light lenses 46, 48 in close proximity around the objective lens 34. This enhances the ability of the white light lenses 42, 44 and the ultraviolet light lenses 46, 48 to provide a maximized amount of illumination to the environment being viewed by the objective lens 34.

There is also a yellow light emitting lens or a yellow light lens 52 provided on the distal end surface 32. The yellow light lens 52 is positioned on the distal end surface 32 along the second center line CL2 and on the opposite side of the first center line CL1 from the objective lens 34, from the pair of white light lenses 42, 44 and from the pair of ultraviolet light lenses 46, 48. The yellow light lens 52 also has an exterior surface with a convex or domed configuration. The convex or domed configuration of the yellow light lens 52 is comparatively greater than any convex configuration of the objective lens 34. The yellow light lens 52 is provided to illuminate areas of the environment adjacent to the distal end surface 32 being observed by the objective lens 34 that may not otherwise be illuminated by the white light lenses 42, 44 or the ultraviolet light lenses 46, 48.

Both of the white light lenses 42, 44, both of the ultraviolet light lenses 46, 48 and the yellow light lens 52 are controllable to retract flush with or behind the distal end surface 32 when not in use, and to extend from the distal end surface 32 when in use.

As various modifications could be made in the construction of the endoscope of this disclosure and its method of operation herein described and illustrated without departing from the scope of the invention, it is intended that all matter contained in the foregoing description or shown in the accompanying drawings shall be interpreted as illustrative rather than limiting. Thus, the breadth and scope of the present disclosure should not be limited by any of the above described exemplary embodiments but should be defined only in accordance with the following claims appended hereto and their equivalents.

1. An endoscope comprising:
an insertion tube, the insertion tube having a flexible length;
a distal tip at one end of the flexible length of the insertion tube, the distal tip having a distal end surface, the distal end surface having a circular configuration;
an objective lens on the distal end surface;
a white light lens on the distal end surface;
an ultraviolet light lens on the distal end surface; and
a yellow light lens on the distal end surface.

2. The endoscope of claim 1, further comprising:
the white light lens having an exterior surface with a convex configuration.

3. The endoscope of claim 2, further comprising:
the white light lens is one of two white light lenses on the distal surface; and
the two white light lenses have exterior surfaces with convex configurations.

4. The endoscope of claim 3, further comprising:
the distal end surface having a center line that bisects the distal end surface; and
the two white light lenses are positioned on the distal end surface on opposite sides of the center line.

5. The endoscope of claim 1, further comprising:
the ultraviolet light lens having an exterior surface with a convex configuration.

6. The endoscope of claim 5, further comprising:
the ultraviolet light lens is one of two ultraviolet light lenses on the distal end surface; and
the two ultraviolet light lenses have exterior surfaces with convex configurations.

7. The endoscope of claim 6, further comprising:
the distal end surface having a center line that bisects the distal end surface; and
the two ultraviolet light lenses are positioned on the distal end surface on opposite sides of the center line.

8. The endoscope of claim 1, further comprising:
the yellow light lens having an exterior surface with a convex configuration.

9. The endoscope of claim 8, further comprising;
the distal end surface having a center line that passes through the objective lens; and
the yellow light lens is positioned on the distal end surface with the center line passing through the yellow light lens.

10. The endoscope of claim 1, further comprising:
the white light lens is one of two white light lenses on the distal end surface;
the two white light lenses have exterior surfaces with convex configurations;
the ultraviolet light lens is one of two ultraviolet light lenses on the distal end surface;
the two ultraviolet light lenses have exterior surfaces with convex configurations; and
the yellow light lens has an exterior surface with a convex configuration.

11. The endoscope of claim 10, further comprising:
the two white light lenses, the two ultraviolet light lenses, and the yellow light lens are controllable to retract behind the distal end surface when not in use, and are controllable to extend from the distal end surface when in use.

12. An endoscope comprising:
a distal end tip of the endoscope, the distal end tip having a distal end surface, the distal end surface having a circular configuration with a center point of the circular configuration;
an objective lens on the distal end surface, the objective lens being positioned off center on the distal end surface with the objective lens being spaced to a side of the center point of the circular configuration; and
a pair of ultraviolet light lenses on the distal end surface, the pair of ultraviolet light lenses being positioned off center on the distal end surface with the pair of ultraviolet light lenses being spaced to one side of the center point of the circular configuration.

13. The endoscope of claim 12, further comprising:
the circular configuration of the distal end surface has a center line that extends across the distal end surface and through the center point; and
the objective lens and the pair of ultraviolet lenses are all positioned off center on the distal end surface and are all spaced to a same side of the center line on the distal end surface.

14. The endoscope of claim 12, further comprising:
a pair of white light lenses on the distal end surface, the pair of white light lenses being positioned off center on the distal end surface with the pair of white light lenses being spaced to a side of the center point of the circular configuration.

15. The endoscope of claim 14, further comprising:
the circular configuration of the distal end surface has a center line that extends across the distal end surface and through the center point; and
the objective lens, the pair of ultraviolet lenses and the pair of white light lenses are all positioned off center on the distal end surface and are all spaced to a same side of the center line on the distal end surface.

16. The endoscope of claim 12, further comprising:
a yellow light lens on the distal end surface, the yellow light lens being positioned off center on the distal end surface with the yellow light lens being spaced on an opposite side of the center point of the circular configuration from the objective lens.

17. The endoscope of claim 16, further comprising:
the circular configuration of the distal end surface has a center line that extends across the distal end surface and through the center point; and the objective lens and the yellow light lens are both positioned on the distal end surface along the center line and are centered on the center line.

18. An endoscope comprising:
a distal end tip of the endoscope, the distal end tip having a distal end surface, the distal end surface having a circular configuration with a center point of the circular configuration;
an objective lens on the distal end surface, the objective lens being positioned off center on the distal end surface with the objective lens being spaced to one side of the center point of the circular configuration;
a pair of white light lenses on the distal end surface, the pair of white light lenses being positioned off center on the distal end surface with the pair of white light lenses being spaced to one side of the center point of the circular configuration; and
a pair of ultraviolet light lenses on the distal end surface, the pair of ultraviolet light lenses being positioned off center on the distal end surface with the pair of ultraviolet light lenses being spaced to one side of the center point of the circular configuration.

19. The endoscope of claim 18, further comprising:
the circular configuration of the distal end surface having a center line that extends across the distal end surface and through the center point; and
the objective lens, the pair of white light lenses and the pair of ultraviolet light lenses are all positioned off center on the distal end surface and are all spaced to a same side of the center line on the distal end surface.

20. The endoscope of claim 19, further comprising:
the pair of white light lenses and the pair of ultraviolet light lenses have exterior surfaces with convex configurations.
